(19) United States
(12) Plant Patent Application Publication
(10) Pub. No.: US 2025/0331435 P1
(43) Pub. Date: Oct. 23, 2025

(54) GUAYULE PLANT NAMED E-B2WN-13167

(50) ;

(71) Applicant: Bridgestone Corporation, Chuo-ku (JP)

(72) Inventors: Von Mark Vicente Cruz, Casa Grande, AZ (US); David Allen Dierig, Phoenix, AZ (US)

(73) Assignee: Bridgestone Corporation, Chuo-ku (JP)

(21) Appl. No.: 19/187,105

(22) Filed: Apr. 23, 2025

Related U.S. Application Data
(60) Provisional application No. 63/637,778, filed on Apr. 23, 2024.

(51) Int. Cl. A01H 6/00; A01H 5/02; A01H 5/12

(52) U.S. Cl. CPC A01H 6/00; A01H 5/02; A01H 5/12
USPC PLT/226

(57) ABSTRACT

A new and distinct cultivar of Parthenium argentatum named E-B2WN-13167 is disclosed, characterized by reduced internode lengths, shorter leaf peduncles, thicker leaves, lower number of flowers, aborted seed production, and slower growth.

### CROSS-REFERENCE TO RELATED APPLICATIONS

This application claims the benefit of U.S. Provisional Application Ser. No. 63/637,778, filed Apr. 23, 2024, the contents of which are incorporated in their entirety herein.

Genus and species: Parthenium argentatum.

Variety denomination: E-B 2WN-13167.

### BACKGROUND OF THE NEW PLANT

The present invention relates to a new and distinct variety of guayule, botanically known as Parthenium argentatum and hereinafter referred to by the variety name “E-B2WN-13167.” E-B2WN-13167 originated from controlled irradiation of seeds from the Parthenium argentatum USDA germplasm line AZ-2 (PI 599675), unpatented.

In 2016, a bulk seed lot of germplasm from AZ-2 was subjected to fast neutron irradiation. The seeds were sown and plants were grown for evaluation in Applicant's research facility located in Eloy, Arizona. A sexual reproduction of E-B2WN-13167 was performed by the propagation of vegetative cuttings. Multiple generations have since been produced and have shown that the unique features of this cultivar are stable and reproduced true-to-type in successive generations.

### SUMMARY

The following characteristics of the new cultivar have been repeatedly observed and can be used to distinguish E-B2WN-13167 as a new and distinct cultivar of Parthenium argentatum plant.

The E-B2WN-13167 cultivar has not been observed and evaluated under all possible conditions and phenotypic differences may be observed with variations in environmental, climatic, and cultural conditions, however, without any variance in genotype.

Plants of the E-B2WN-13167 cultivar can be distinguished from the AZ-2 variety in several characteristics. For example, plants of the E-B2WN-13167 cultivar have reduced internode lengths, shorter leaf peduncles, thicker leaves, lower numbers of flowers, and exhibit slower growth compared with the AZ-2. The E-B2WN-13167 cultivar has aborted seed production compared with the AZ-2 variety.

### BRIEF DESCRIPTION OF THE DRAWINGS

The accompanying color photographs illustrate the overall appearance of E-B2WN-13167 showing the distinguishing characteristics as true as is reasonably possible to obtain in color reproductions of this type.

FIG. 1 is a photograph depicting the overall growth habit of the E-B2WN-13167 cultivar.

FIG. 2 is a photograph depicting the smaller flowers of the E-B2WN-13167 cultivar.

FIG. 3 is a photograph depicting the leaves of the E-B2WN-13167 cultivar.

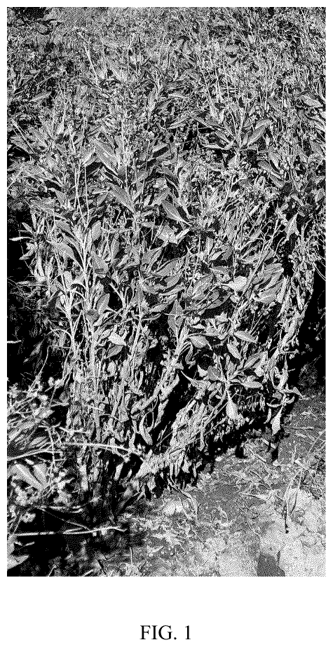

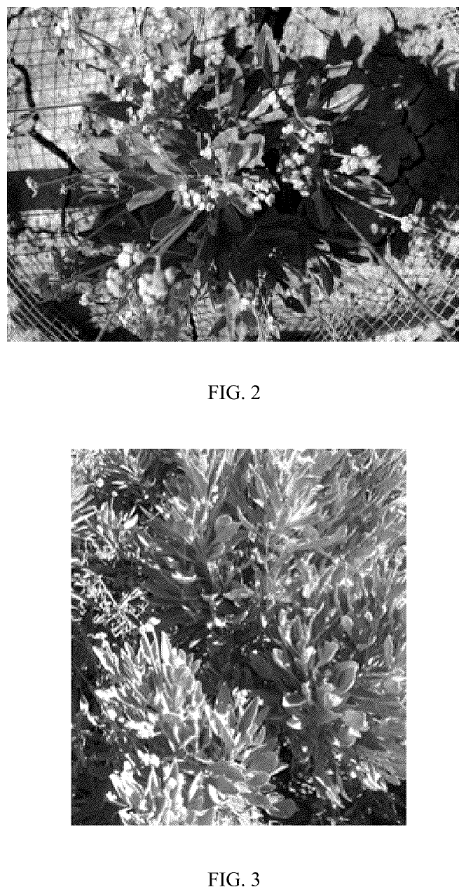

### DETAILED BOTANICAL DESCRIPTION OF THE PLANT

The following is a detailed botanical description of the E-B2WN-13167 cultivar. Observations, measurements, values, and comparisons were collected at Applicant's research facility in Eloy, Arizona.

The E-B2WN-13167 cultivar has shorter internodes resulting in higher numbers of leaves (37±3) per 10 cm branch section compared with AZ-2 (23±2). The internode length of the E-B2WN-13167 cultivar has a length of 0.6±0.23 cm on branches with 0.40 cm branch diameter and 0.72±0.26 cm on branches with 0.70 cm branch diameter, compared with the AZ-2 having an internode length of 0.70±0.25 cm on branches with 0.40 cm diameter and 1.27±0.45 cm on branches with 0.70 cm diameter.

The leaves of the E-B2WN-13167 cultivar have relatively short peduncles (5.01±1.66 cm) compared to other AZ-2 shrubs of the same age (17.62±3.68 cm). The E-B2WN-13167 cultivar had fewer flowers during the growing period, with a smaller flower diameter (0.65±0.02 cm) compared to the wild type (0.82±0.02 cm). The E-B2WN-13167 cultivar has a smaller leaf area (5.07±0.35 cm²) compared to the AZ-2 (5.56±0.73 cm²). The E-B2WN-13167 cultivar is smaller than the AZ-2 and shows a slower rate of growth (0.06 cm/day) compared to the AZ-2 variety (0.1 cm/day).
Botanical classification:
   i. Family.—Asteraceae.
   ii. Genus.—Parthenium.
   iii. Species.—argentatum.
   iv. Common name.—Guayule.
   v. Duration.—Perennial.
Parentage: i. Controlled irradiation of seeds from the Parthenium argentatum USDA germplasm line AZ-2 (PI 599675), unpatented.
Propagation:
   i. Type.—Vegetative cuttings.
   ii. Success of rooting.—Approximately 20% under standard soil cutting propagation practices.
   iii. Rooting system.—Deep, coarse taproot and a shallow fibrous system
   iv. Seeds.—No viable seed observed. Low seed production.
Plant characteristics:
   i. Height.—55 cm at 2 years, 70 cm at 4 years.
   ii. Width.—28 cm at 2 years, 50 cm at 4 years.
   iii. Commercial crop time.—2 years.
   iv. Growth habit.—Shrub with thick lignified stems, and a low, spreading form.
   v. Growth rate.—0.06 cm/day.
   vi. Internode length.—0.65±0.23 cm on 0.40 cm branch diameter. 0.72±0.26 cm on 0.70 cm branch diameter.
Foliage description:
   i. Quantity of leaves.—37±3 per 10 cm section of branch.
   ii. Type of leaves.—Densely haired leaves covered with white wax.
   iii. Leaf shape.—Lanceolate to oblanceolate.
   iv. Margin.—Serrated.
   v. Length of leaf.—3.46±1.2 cm.
   vi. Width of leaf.—0.78±0.2 cm.
   vii. Leaf area.—5.07±0.35 cm².
   viii. Color.—Green.
Flowering:
   i. Flowering habit.—Indeterminate.
   ii. General inflorescence.—1. Flower diameter: 0.65±0.02 cm. 2. Color: White petals.
   iii. Peduncle.—1. Length: 5.01±1.66 cm.
Use: Commercial production of natural rubber, ethanol, non-toxic adhesives, and hypoallergenic latex.
Rubber production: Rubber content after two years was 3.58% relative to biomass.
Fecundity:
   i. Seed production.—No viable seeds.
Disease and pest resistance: Resistant to many pests and diseases due to production of terpene resins.

What is claimed is.—

1. A new and distinct cultivar of Parthenium argentatum plant named E-B2WN-13167, substantially as described and illustrated herein.
